(19) United States
(12) Patent Application Publication
(10) Pub. No.: US 2020/0238034 A1
(43) Pub. Date: Jul. 30, 2020

(54) LARYNGEAL MASK AIRWAY DEVICE

(71) Applicant: CHIH HUNG SHAN, YILAN COUNTY (TW)

(72) Inventor: CHIH HUNG SHAN, YILAN COUNTY (TW)

(21) Appl. No.: 16/749,196

(22) Filed: Jan. 22, 2020

(30) Foreign Application Priority Data
Jan. 25, 2019 (TW) 108201262

(51) Int. Cl. A61M 16/04; A61M 16/20

(52) U.S. Cl. CPC A61M 16/0447; A61M 2205/0216; A61M 16/0486; A61M 16/208

(57) ABSTRACT

A laryngeal mask airway device includes a flexible tube set and an annular mask. The flexible tube set has an airway tube and an air guide tube that are juxtaposed. The annular mask has an outer membrane surrounding an outer periphery of one end of the airway tube and an elastic filler disposed inside the outer membrane. The outer membrane is in communication with one end of the air guide tube. When in use, the outer membrane is first squeezed to compress the elastic filler, and then the other end of the air guide tube is closed. The inside of the outer membrane forms a negative pressure to maintain the elastic filler in a small volume, so that one end having the annular mask of the flexible tube set can be placed and positioned in a patient's throat.

### FIELD OF THE INVENTION

The present invention relates to a laryngeal mask airway device, and more particularly to a laryngeal mask airway device that can be operated and used easily and that allows an inflated mask to maintain proper soft and hard support, thereby achieving an effective sealing effect and reducing the patient's discomfort.

### BACKGROUND OF THE INVENTION

In these days, in clinical medicine, there is often a need for surgical anesthesia or other medical gases to be introduced into the human body. A common way is to use an air guide tube and an endotracheal tube for intubation to maintain the smooth flow of the airway during anesthesia or the introduction of medical gases into the human body. However, many patients may suffer great discomfort because the actions of the intubation described above. In order to alleviate the suffering of patients, another laryngeal mask airway device instead of the tracheal intubation is gradually being widely used in the need to maintain the smoothness of the airway.

Taiwanese Utility Model Publication No. M340819 discloses a self-assembled laryngeal mask airway device, which mainly comprises a gas supply mask unit and an auxiliary member. The gas supply mask unit includes a main seat tube that can be assembled and disassembled, a support pad, a positioning piece, and a fitting membrane. The main seat tube includes a tube wall surrounding an axis and defining a perforation, a limiting piece extending radially outward from the periphery of the distal end of the tube wall, and an outward enlarged body located above the limiting piece and extending outwardly from the outer circumferential surface of the tube wall. The support pad is detachably disposed between the outward enlarged body of the main seat tube and the limiting piece. The support pad includes a top surface facing the outward enlarged body, a bottom surface facing the limiting piece, and a positioning hole that extends from the top surface to the bottom surface and is disposed between the limiting piece and the outward enlarged body. The positioning piece is attached to the bottom surface of the support pad. The positioning piece includes a body portion corresponding to the support pad, and a mounting portion disposed along the periphery of the top surface of the body portion for coupling with the support pad. The body portion has a first vent and a second vent spaced apart from each other and corresponding to the through hole of the main seat tube. The fitting membrane is wrapped around the outward enlarged body and the limiting piece of the main seat tube, the support pad and the positioning piece. The fitting membrane is formed with a hole for insertion of the tube wall of the main seat tube and an opening that is spaced apart from the hole and corresponds to the first and second vents of the positioning piece. The auxiliary member is inserted into the fitting membrane of the air supply mask unit for assisting the air supply mask unit to further advance toward the throat of a patient. In the above structure, since the support pad is directly covered by the fitting membrane, the fitting membrane is supported by the support pad to have a normal inflated shape. Therefore, when in use, the fitting membrane is inserted into the throat in a fully inflated state. Although the support pad is squeezed and elastically deformed during the process of extending into the throat, it still causes extreme discomfort to the patient.

Taiwanese Utility Model Publication No. M567115 discloses a laryngeal mask airway device, comprising a mask and a tubular body. The mask has a bladder and a support member. The top of the support member has a groove. The bladder is correspondingly disposed on the groove. The tubular body has a ventilation tube and a filling tube in communication with the bladder. One end of the tubular body is connected with the support member, and the joint portion thereof has an opening communicating with one end of the ventilation tube. The filling tube is configured to inject an external filler (which may be a liquid or a gas) into the bladder so that the bladder is fully inflated.

In such a structure, the filler (which may be a liquid or a gas) is filled into the bladder via the filling tube, so that the bladder is slightly inflated by the thrust of the filler from the inside to the outside to generate inflation slightly greater than that in a natural state, thereby attaching to the inner wall of the throat to close the esophagus and maintain the smoothness of the airway. However, since the inflation of the bladder cannot be directly observed when the bladder is inserted into the throat, the filler of the bladder may be insufficient (the bladder is too soft, the sealing effect is not good) or the filler of the bladder may be excessive (the bladder is too hard, causing the patient to be extremely unwell).

Accordingly, the inventor of the present invention has devoted himself based on his many years of practical experiences to solve these problems.

### SUMMARY OF THE INVENTION

The primary object of the present invention is to provide a laryngeal mask airway device. One end of an airway tube is provided with an annular mask. The annular mask has an outer membrane surrounding the outer periphery of the end of the airway tube and an elastic filler that is disposed inside the outer membrane and provides an outward supporting force. The outer membrane is communicated with the outside through an air guide tube. When the outer membrane is compressed to remove the internal fluid, the air guide tube is closed, so that the outer membrane and the elastic filler are compressed to maintain a small volume. A negative pressure is formed inside the outer membrane, so that one end having the annular mask of the airway tube can be placed into the patient's throat easily. When the air guide tube is opened to the outside, the pressure inside and outside the outer membrane can be balanced, so that the annular mask returns to its original size and shape by its own elasticity. The outer membrane is supported by the inflation of the elastic filler to generate a slight outward inflation, so that the outer membrane can be more closely pressed against the inner wall of the throat, and the annular mask can cover the esophagus tightly and keep the airway open.

Another object of the present invention is to provide a laryngeal mask airway device. An intake and exhaust assembly is disposed at one end of the air guide tube away from the annular mask. The intake and exhaust assembly includes an exhaust valve and an intake valve that are in communication with the air guide tube, and a plug for blocking the intake valve. When the plug blocks the air inlet, the air in the outer membrane can be exhausted via the exhaust valve, and the plug is removed, and the outside air can be unidirectionally introduced into the outer membrane via the intake valve, thereby providing an intake and exhaust mechanism that can be operated simply and controlled conveniently.

In order to achieve the above objects and effects, a laryngeal mask airway device is provided. The laryngeal mask airway device comprises a flexible tube set and an annular mask. The flexible tube set has an airway tube and an air guide tube that are juxtaposed. The annular mask has an outer membrane surrounding an outer periphery of one end of the airway tube and an elastic filler disposed inside the outer membrane. The outer membrane is in communication with one end of the air guide tube. Another end of the air guide tube is openable and closable. The outer membrane can be inflated and supported by the elastic filler in a natural state so that the annular mask provides an outward elastic supporting force greater than the annular mask itself.

Preferably, an intake and exhaust assembly is disposed at the end of the air guide tube away from the outer membrane. The intake and exhaust assembly includes an exhaust valve, an intake valve, and a plug. The exhaust valve has a unidirectional air outlet. The intake valve has a unidirectional air inlet. The plug is configured to close the air inlet.

Preferably, one end of the flexible tube set, away from the annular mask, is transversely provided with an end plate. One side of the end plate, close to the air guide tube, is recessed with a clamping groove. When the air guide tube is embedded in the clamping groove, the air guide tube is pressed to form a closed state.

Preferably, the clamping groove is provided with a clamping recess close to an edge of the end plate for holding the air guide tube so that the air guide tube can be kept and positioned in the clamping recess.

Preferably, the flexible tube set further includes an extraction tube, and one end of the extraction tube is formed with a front opening on a surface of the outer membrane.

Preferably, the extraction tube and a part of the air guide tube are integrally formed at two sides of the airway tube.

Preferably, the elastic filler is a composite material layer that is formed inside the outer membrane and has an outward elastic tension.

Preferably, the elastic filler is an elastic thin layer having an outward supporting force against an inner surface of the outer membrane.

Preferably, the elastic filler is made of a foam material.

Embodiments of the present invention will now be described, by way of example only, with reference to the accompanying drawings.

### BRIEF DESCRIPTION OF THE DRAWINGS

FIG. 1 is a perspective view of the present invention;

FIG. 2 is a schematic view showing the operation that the annular mask of the present invention is compressed to reduce its volume;

FIG. 3 is a schematic view showing a state in which the annular mask of the present invention is kept small;

FIG. 4 is a schematic view showing the operation that the annular mask of the present invention is returned to its original shape from a compressed state;

FIG. 5 is a schematic view showing a state in which the laryngeal mask airway device of the present invention is positioned in the throat;

FIG. 6 is a schematic view showing a state in which the air guide tube is held and positioned by the end plate in accordance with a first embodiment of the present invention;

FIG. 7 is a schematic view showing a state in which the air guide tube is held and closed by the end plate in accordance with the first embodiment of the present invention; and

FIG. 8 is a schematic view showing a state in which the air guide tube is held and closed by the end plate in accordance with a second embodiment of the present invention.

### DETAILED DESCRIPTION OF THE PREFERRED EMBODIMENTS

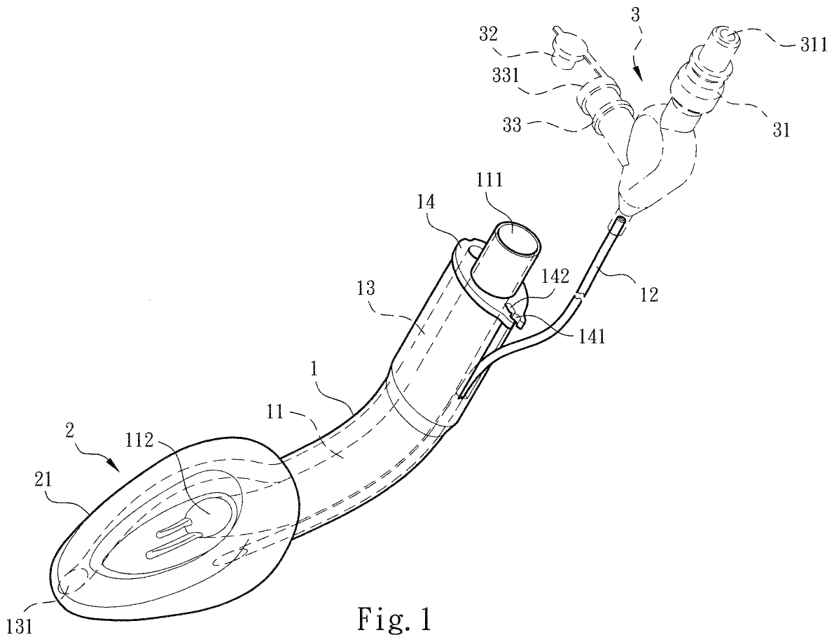

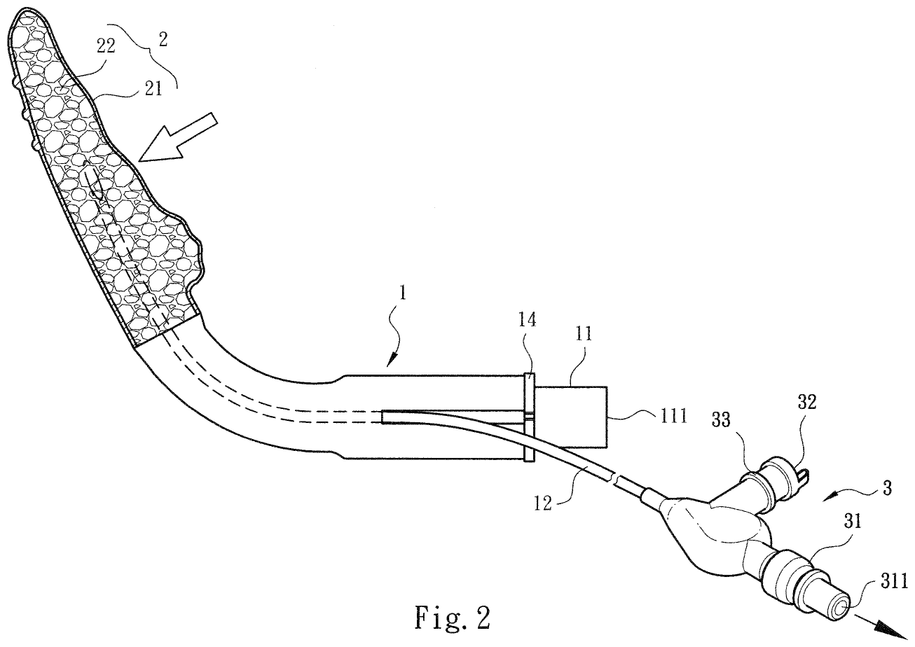

Referring to FIG. 1 and FIG. 2, the present invention mainly comprises a flexible tube set 1 and an annular mask 2. The flexible tube set 1 includes an airway tube 11 and an air guide tube 12 that are juxtaposed. The airway tube 11 has two ends defined as a first opening 111 and a second opening 112.

The annular mask 2 has an outer membrane 21 surrounding the outer periphery of the second opening 112 of the airway tube 11 and an elastic filler 22 disposed inside the outer membrane 21. The inside of the outer membrane 21 is in communication with one end of the air guide tube 12, and another end of the air guide tube 12 is openable and closable.

An intake and exhaust assembly 3 is disposed at one end of the air guide tube 12 away from the outer membrane 21 as one mechanism that the end can be closed or opened. The intake and exhaust assembly 3 includes an exhaust valve 31, a plug 32, and an intake valve 33. The exhaust valve 31 has a unidirectional air outlet 311. The intake valve 33 has a unidirectional air inlet 331. The plug 32 is configured to close the air inlet 331.

In this embodiment, the elastic filler 22 is made of a foam material. The elastic filler 22 can be compressed into a smaller volume when subjected to an external pressure. When the elastic filler 22 is in a natural state, it can support the outer membrane 21 uniformly to be fully inflated.

In a feasible embodiment, the flexible tube set 1 further includes an extraction tube 13. One end of the extraction tube 3 is formed with a front opening 131 on the surface of the outer membrane 21 for extracting the liquid of the esophagus and the stomach. The extraction tube 13 and a part of the air guide tube 12 are integrally formed at two sides of the airway tube 11 to form a simple integrated structure.

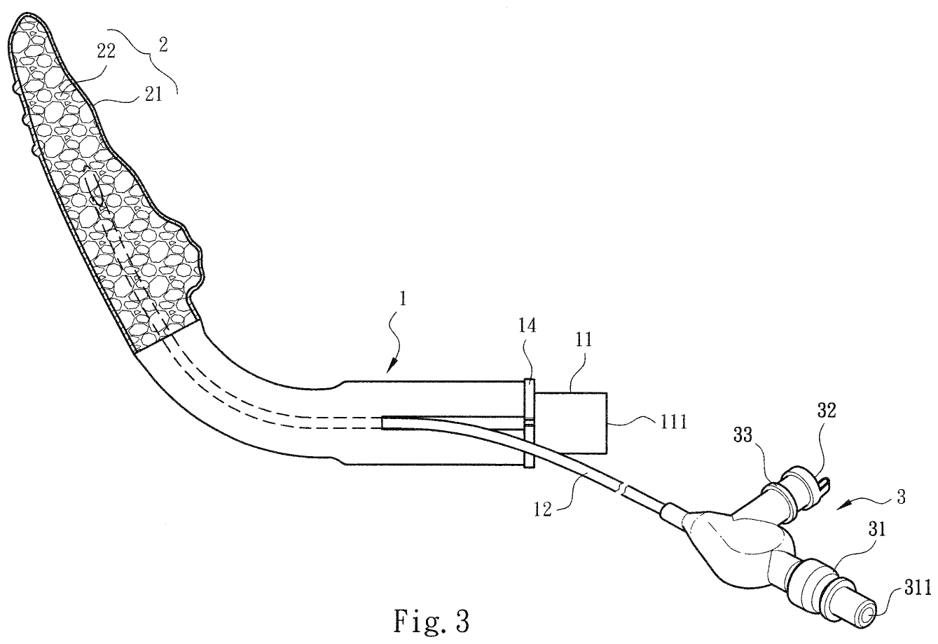

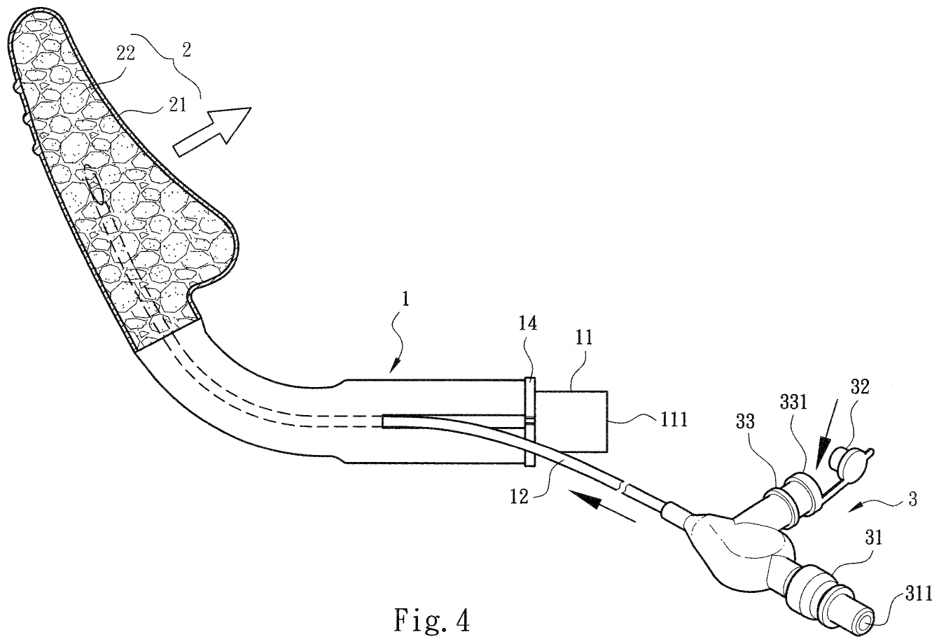

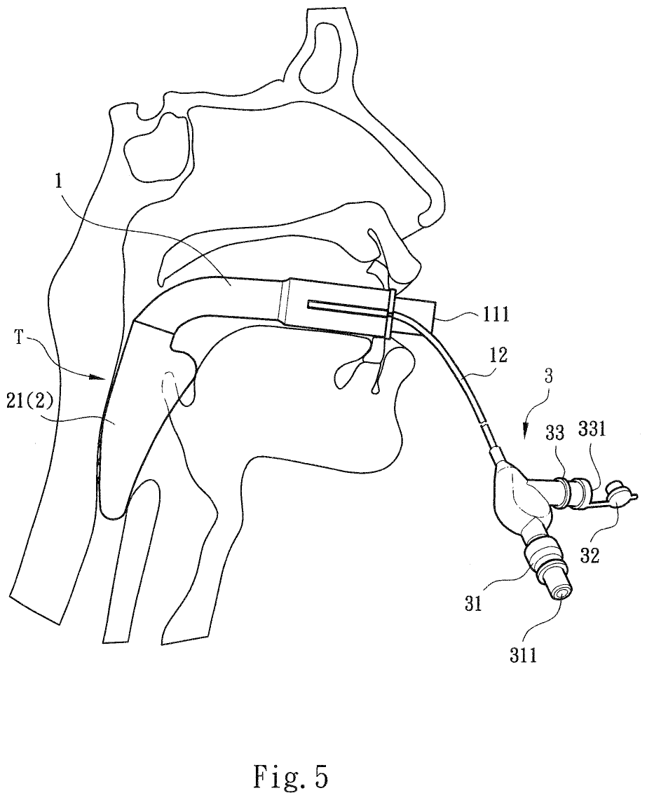

As shown in FIG. 3 to FIG. 5, when in use, in a state that the plug 32 closes the air inlet 331, the outer membrane 21 is first squeezed to compress the elastic filler 22 to reduce its volume, and the air in the outer membrane 21 can be exhausted from the air outlet 311 of the exhaust valve 31 via the air guide tube 12 (as shown in FIG. 2). Since the outside air cannot be introduced into the outer membrane 21 via the air inlet 331, a negative pressure is formed inside the outer membrane 21, and the elastic filler 22 can be kept in a compressed state to reduce its volume (as shown in FIG. 3), so that one end having the annular mask 2 of the airway tube 11 can be placed into the patient's throat T easily. When the annular mask 2 is placed and positioned inside the throat T, the plug 32 is removed from the air inlet 331 so that the external air is introduced unidirectionally through the air inlet 331 and enters the outer membrane 21 through the air guide tube 12 (as shown in FIG. 4) to balance the air pressure inside and outside the outer membrane 21. At this time, the elastic filler 22 is naturally inflated because of not being applied with a force. In addition to supporting the outer membrane 21 (the annular mask 2) to return to the original size and shape, the elastic filler 22 provides a soft pressing force from the inside of the outer membrane 21 toward the outside of the outer membrane 21, assisting the outer membrane 21 to generate re-inflation slightly greater than that in a natural state, so that the outer surface of the outer membrane 21 is more closely pressed against the inner wall of the esophageal opening, and the annular mask 2 covers the esophagus effectively and keeps the airway open (as shown in FIG. 5).

In the above structure of the present invention, since the elastic filler 22 is made of a foam material or other soft material, after being fully inflated, the elastic filler 22 allows the annular mask 2 to attach to the inner wall of the esophageal opening than the natural inflation of the outer membrane 21 itself, while still maintaining proper softness and elasticity. In this way, the undesired discomfort of the patient's throat T caused by excessively increasing the internal air pressure of the annular mask 2 can be alleviated effectively.

In the actual application, the elastic filler 22 may be a composite material layer that is formed inside the outer membrane 21 and has an outward elastic tension. The elastic filler 22 may also be an elastic thin layer having an outward supporting force against the inner surface of the outer membrane 21 from the inside to the outside. Both can achieve the effects of reducing the volume when the outer membrane 21 is compressed, and assisting the outer membrane 21 in generating inflation slightly greater than that in a natural state after being released, and forming elastic support.

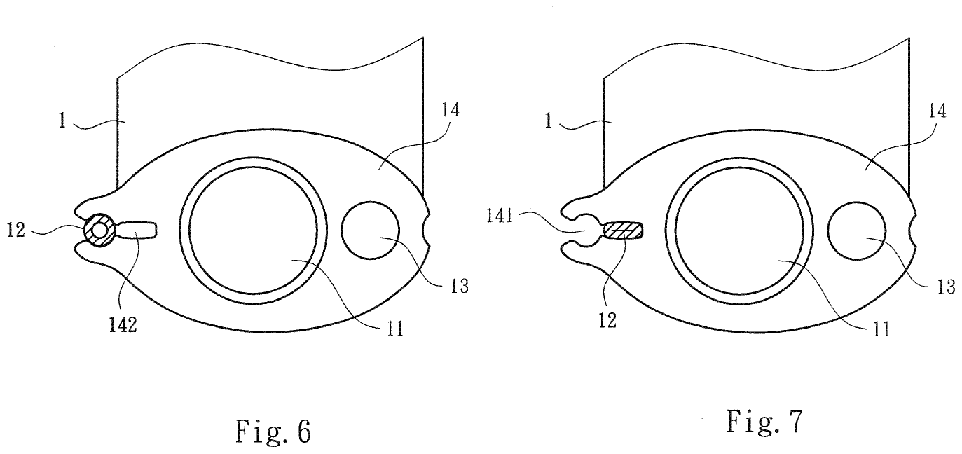

Referring to FIG. 6 and FIG. 7, in the actual application, one end of the flexible tube set 1, away from the annular mask 2, may be transversely provided with an end plate 14 as needed. One side of the end plate 14, close to the air guide tube 12, is recessed with a clamping groove 142. The clamping groove 142 is provided with a clamping recess 141 close to the edge of the end plate 14. The air guide tube 12 is hold by the clamping recess 141, so that the air guide tube 12 can be kept and positioned in the clamping recess 141. When the air guide tube 12 is further embedded in the clamping groove 142, the wall of the middle portion of the air guide tube 12 is pressed to form a fit, thereby closing the air guide tube 12.

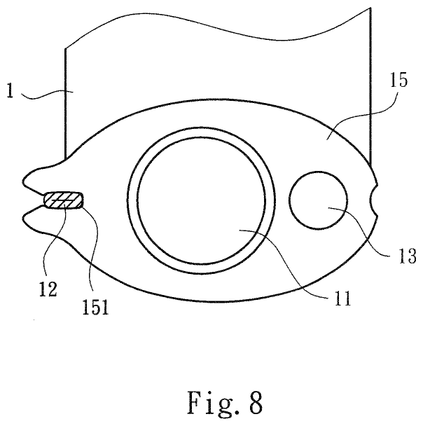

As shown in FIG. 8, in another feasible embodiment, one end of the flexible tube set 1, away from the annular mask 2, may be transversely provided with an end plate 15 as needed. One side of the end plate 15, close to the air guide tube 12, is recessed with a clamping groove 151. When the air guide tube 12 is embedded in the clamping groove 151, the wall of the middle portion of the air guide tube 12 is pressed to form a fit. Compared with the above-mentioned end plate 14, although the end plate 15 does not have a structure similar to the clamping recess 141, the effect of closing the air guide tube 12 can be achieved.

In summary, the improved laryngeal mask airway device of the present invention can achieve easy operation, proper soft and hard support after being inflated, and can have an excellent sealing effect and reduce patient discomfort. Although particular embodiments of the present invention have been described in detail for purposes of illustration, various modifications and enhancements may be made without departing from the spirit and scope of the present invention. Accordingly, the present invention is not to be limited except as by the appended claims.

What is claimed is:

1. A laryngeal mask airway device, comprising:
a flexible tube set, having an airway tube and an air guide tube that are juxtaposed;
an annular mask, having an outer membrane surrounding an outer periphery of one end of the airway tube and an elastic filler disposed inside the outer membrane, the outer membrane being in communication with one end of the air guide tube, another end of the air guide tube being openable and closable, wherein the outer membrane can be inflated and supported by the elastic filler in a natural state, so that the annular mask provides an outward elastic supporting force greater than the annular mask itself.

2. The laryngeal mask airway device as claimed in claim 1, wherein the elastic filler is made of a foam material.

3. The laryngeal mask airway device as claimed in claim 1, wherein the elastic filler is a composite material layer that is formed inside the outer membrane and has an outward elastic tension.

4. The laryngeal mask airway device as claimed in claim 1, wherein the elastic filler is an elastic thin layer having an outward supporting force against an inner surface of the outer membrane.

5. The laryngeal mask airway device as claimed in claim 1, wherein an intake and exhaust assembly is disposed at the end of the air guide tube away from the outer membrane, the intake and exhaust assembly includes an exhaust valve, an intake valve and a plug, the exhaust valve has a unidirectional air outlet, the intake valve has a unidirectional air inlet, and the plug is configured to close the air inlet.

6. The laryngeal mask airway device as claimed in claim 2, wherein an intake and exhaust assembly is disposed at the end of the air guide tube away from the outer membrane, the intake and exhaust assembly includes an exhaust valve, an intake valve and a plug, the exhaust valve has a unidirectional air outlet, the intake valve has a unidirectional air inlet, and the plug is configured to close the air inlet.

7. The laryngeal mask airway device as claimed in claim 3, wherein an intake and exhaust assembly is disposed at the end of the air guide tube away from the outer membrane, the intake and exhaust assembly includes an exhaust valve, an intake valve and a plug, the exhaust valve has a unidirectional air outlet, the intake valve has a unidirectional air inlet, and the plug is configured to close the air inlet.

8. The laryngeal mask airway device as claimed in claim 4, wherein an intake and exhaust assembly is disposed at the end of the air guide tube away from the outer membrane, the intake and exhaust assembly includes an exhaust valve, an intake valve and a plug, the exhaust valve has a unidirectional air outlet, the intake valve has a unidirectional air inlet, and the plug is configured to close the air inlet.

9. The laryngeal mask airway device as claimed in claim 1, wherein one end of the flexible tube set, away from the annular mask, is transversely provided with an end plate, one side of the end plate, close to the air guide tube, is recessed with a clamping groove, when the air guide tube is embedded in the clamping groove, the air guide tube is pressed to form a closed state.

10. The laryngeal mask airway device as claimed in claim 2, wherein one end of the flexible tube set, away from the annular mask, is transversely provided with an end plate, one side of the end plate, close to the air guide tube, is recessed with a clamping groove, when the air guide tube is embedded in the clamping groove, the air guide tube is pressed to form a closed state.

11. The laryngeal mask airway device as claimed in claim 3, wherein one end of the flexible tube set, away from the annular mask, is transversely provided with an end plate, one side of the end plate, close to the air guide tube, is recessed with a clamping groove, when the air guide tube is embedded in the clamping groove, the air guide tube is pressed to form a closed state.

12. The laryngeal mask airway device as claimed in claim 4, wherein one end of the flexible tube set, away from the annular mask, is transversely provided with an end plate, one side of the end plate, close to the air guide tube, is recessed with a clamping groove, when the air guide tube is embedded in the clamping groove, the air guide tube is pressed to form a closed state.

13. The laryngeal mask airway device as claimed in claim 5, wherein one end of the flexible tube set, away from the annular mask, is transversely provided with an end plate, one side of the end plate, close to the air guide tube, is recessed with a clamping groove, when the air guide tube is embedded in the clamping groove, the air guide tube is pressed to form a closed state.

14. The laryngeal mask airway device as claimed in claim 6, wherein one end of the flexible tube set, away from the annular mask, is transversely provided with an end plate, one side of the end plate, close to the air guide tube, is recessed with a clamping groove, when the air guide tube is embedded in the clamping groove, the air guide tube is pressed to form a closed state.

15. The laryngeal mask airway device as claimed in claim 7, wherein one end of the flexible tube set, away from the annular mask, is transversely provided with an end plate, one side of the end plate, close to the air guide tube, is recessed with a clamping groove, when the air guide tube is embedded in the clamping groove, the air guide tube is pressed to form a closed state.

16. The laryngeal mask airway device as claimed in claim 8, wherein one end of the flexible tube set, away from the annular mask, is transversely provided with an end plate, one side of the end plate, close to the air guide tube, is recessed with a clamping groove, when the air guide tube is embedded in the clamping groove, the air guide tube is pressed to form a closed state.

17. The laryngeal mask airway device as claimed in claim 9, wherein the clamping groove is provided with a clamping recess close to an edge of the end plate for holding the air guide tube so that the air guide tube can be kept and positioned in the clamping recess.

18. The laryngeal mask airway device as claimed in claim 10, wherein the clamping groove is provided with a clamping recess close to an edge of the end plate for holding the air guide tube so that the air guide tube can be kept and positioned in the clamping recess.

19. The laryngeal mask airway device as claimed in claim 11, wherein the clamping groove is provided with a clamping recess close to an edge of the end plate for holding the air guide tube so that the air guide tube can be kept and positioned in the clamping recess.

20. The laryngeal mask airway device as claimed in claim 12, wherein the clamping groove is provided with a clamping recess close to an edge of the end plate for holding the air guide tube so that the air guide tube can be kept and positioned in the clamping recess.

21. The laryngeal mask airway device as claimed in claim 13, wherein the clamping groove is provided with a clamping recess close to an edge of the end plate for holding the air guide tube so that the air guide tube can be kept and positioned in the clamping recess.

22. The laryngeal mask airway device as claimed in claim 14, wherein the clamping groove is provided with a clamping recess close to an edge of the end plate for holding the air guide tube so that the air guide tube can be kept and positioned in the clamping recess.

23. The laryngeal mask airway device as claimed in claim 15, wherein the clamping groove is provided with a clamping recess close to an edge of the end plate for holding the air guide tube so that the air guide tube can be kept and positioned in the clamping recess.

24. The laryngeal mask airway device as claimed in claim 16, wherein the clamping groove is provided with a clamping recess close to an edge of the end plate for holding the air guide tube so that the air guide tube can be kept and positioned in the clamping recess.

25. The laryngeal mask airway device as claimed in claim 1, wherein the flexible tube set further includes an extraction tube, and one end of the extraction tube is formed with a front opening on a surface of the outer membrane, and the extraction tube and at least a part of the air guide tube are integrally formed at two sides of the airway tube.

26. The laryngeal mask airway device as claimed in claim 2, wherein the flexible tube set further includes an extraction tube, and one end of the extraction tube is formed with a front opening on a surface of the outer membrane, and the extraction tube and at least a part of the air guide tube are integrally formed at two sides of the airway tube.

27. The laryngeal mask airway device as claimed in claim 3, wherein the flexible tube set further includes an extraction tube, and one end of the extraction tube is formed with a front opening on a surface of the outer membrane, and the extraction tube and at least a part of the air guide tube are integrally formed at two sides of the airway tube.

28. The laryngeal mask airway device as claimed in claim 4, wherein the flexible tube set further includes an extraction tube, and one end of the extraction tube is formed with a front opening on a surface of the outer membrane, and the extraction tube and at least a part of the air guide tube are integrally formed at two sides of the airway tube.

29. The laryngeal mask airway device as claimed in claim 5, wherein the flexible tube set further includes an extraction tube, and one end of the extraction tube is formed with a front opening on a surface of the outer membrane, and the extraction tube and at least a part of the air guide tube are integrally formed at two sides of the airway tube.

30. The laryngeal mask airway device as claimed in claim 6, wherein the flexible tube set further includes an extraction tube, and one end of the extraction tube is formed with a front opening on a surface of the outer membrane, and the extraction tube and at least a part of the air guide tube are integrally formed at two sides of the airway tube.

31. The laryngeal mask airway device as claimed in claim 7, wherein the flexible tube set further includes an extraction tube, and one end of the extraction tube is formed with a front opening on a surface of the outer membrane, and the extraction tube and at least a part of the air guide tube are integrally formed at two sides of the airway tube.

32. The laryngeal mask airway device as claimed in claim 8, wherein the flexible tube set further includes an extraction tube, and one end of the extraction tube is formed with a front opening on a surface of the outer membrane, and the extraction tube and at least a part of the air guide tube are integrally formed at two sides of the airway tube.

33. The laryngeal mask airway device as claimed in claim 9, wherein the flexible tube set further includes an extraction tube, and one end of the extraction tube is formed with a front opening on a surface of the outer membrane, and the extraction tube and at least a part of the air guide tube are integrally formed at two sides of the airway tube.

34. The laryngeal mask airway device as claimed in claim 10, wherein the flexible tube set further includes an extraction tube, and one end of the extraction tube is formed with a front opening on a surface of the outer membrane, and the extraction tube and at least a part of the air guide tube are integrally formed at two sides of the airway tube.

35. The laryngeal mask airway device as claimed in claim 11, wherein the flexible tube set further includes an extraction tube, and one end of the extraction tube is formed with a front opening on a surface of the outer membrane, and the extraction tube and at least a part of the air guide tube are integrally formed at two sides of the airway tube.

36. The laryngeal mask airway device as claimed in claim 12, wherein the flexible tube set further includes an extraction tube, and one end of the extraction tube is formed with a front opening on a surface of the outer membrane, and the extraction tube and at least a part of the air guide tube are integrally formed at two sides of the airway tube.

37. The laryngeal mask airway device as claimed in claim 13, wherein the flexible tube set further includes an extraction tube, and one end of the extraction tube is formed with a front opening on a surface of the outer membrane, and the extraction tube and at least a part of the air guide tube are integrally formed at two sides of the airway tube.

38. The laryngeal mask airway device as claimed in claim 14, wherein the flexible tube set further includes an extraction tube, and one end of the extraction tube is formed with a front opening on a surface of the outer membrane, and the extraction tube and at least a part of the air guide tube are integrally formed at two sides of the airway tube.

39. The laryngeal mask airway device as claimed in claim 15, wherein the flexible tube set further includes an extraction tube, and one end of the extraction tube is formed with a front opening on a surface of the outer membrane, and the extraction tube and at least a part of the air guide tube are integrally formed at two sides of the airway tube.

40. The laryngeal mask airway device as claimed in claim 16, wherein the flexible tube set further includes an extraction tube, and one end of the extraction tube is formed with a front opening on a surface of the outer membrane, and the extraction tube and at least a part of the air guide tube are integrally formed at two sides of the airway tube.
